(19) United States
(12) Patent Application Publication
(10) Pub. No.: US 2012/0253147 A1
(43) Pub. Date: Oct. 4, 2012

(54) CALIBRATION METHOD AND ARRANGEMENT AND SENSOR FOR NON-INVASIVELY MEASURING BLOOD CHARACTERISTICS OF A SUBJECT

(75) Inventors: Matti Huiku, Espoo (FI); Antti Tolonen, Tampere (FI); Victor Petrovich Ostroverkhov, Ballston Lake, NY (US); Katja Urpalainen, Vantaa (FI)

(73) Assignee: GENERAL ELECTRIC COMPANY, Schenectady, NY (US)

(21) Appl. No.: 13/076,622

(22) Filed: Mar. 31, 2011

(51) Int. Cl. A61B 5/1455

(52) U.S. Cl. 600/310

(57) ABSTRACT

A calibration method for an apparatus for non-invasively monitoring blood characteristics of a subject is disclosed. The apparatus is provided with a computational model representing a relationship between in-vivo measurement signals obtained from the subject and the blood characteristics. The providing includes employing at least one tissue property variable in the computational model, in which the at least one tissue property variable is indicative of absorption and scattering characteristics of the subject's tissue. An arrangement for determining blood characteristics of a subject and a sensor for the arrangement are also disclosed.

### BACKGROUND OF THE INVENTION

This disclosure relates to a calibration method for an apparatus intended for non-invasively monitoring blood characteristics of a subject. This disclosure also relates to the apparatus, which is typically a pulse oximeter, and to a sensor for the apparatus.

Plethysmography refers to measurement of changes in the sizes and volumes of organs and extremities by measuring changes in blood volume. Photoplethysmography relates to the use of optical signals transmitted through or reflected by blood for monitoring a physiological parameter/variable of a subject. Conventional pulse oximeters use red and infrared photoplethysmographic (PPG) waveforms, i.e. waveforms measured respectively at red and infrared wavelengths, to determine oxygen saturation of pulsatile arterial blood of a subject. The two wavelengths used in a conventional pulse oximeter are typically around 660 nm (red wavelength) and 940 nm (infrared wavelength).

Pulse oximetry is at present the standard of care for continuous monitoring of arterial oxygen saturation (SpO₂). Pulse oximeters provide instantaneous in-vivo measurements of arterial oxygenation, and thereby an early warning of arterial hypoxemia, for example. Pulse oximeters also display the photoplethysmographic waveform, which can be related to tissue blood volume and blood flow, i.e. the blood circulation, at the site of the measurement, typically in finger or ear.

Traditionally, pulse oximeters use the above-mentioned two wavelengths, red and infrared, to determine oxygen saturation. Other parameters that may be determined in a two-wavelength pulse oximeter include pulse rate and peripheral perfusion index (PI), for example. Increasing the number of wavelengths to at least four allows the measurement of total hemoglobin (THb, grams per liter) and different hemoglobin types, such as oxyhemoglobin (HbO₂), deoxyhemoglobin (RHb), carboxyhemoglobin (HbCO), and methemoglobin (MetHb). In practice, a pulse oximeter designed to measure all hemoglobin species may be provided with 4 to 8 wavelengths (i.e. light sources) ranging from around 600 nm up to around 1000 nm.

Light photons propagate in living tissues along random paths, which are statistically determined by the scattering and absorption properties of the medium. As the absorption and scattering efficiencies are wavelength-dependent, the average path lengths are different for each wavelength channel in all spectro-photometric devices, such as pulse oximeters. In multi-wavelength oximetry designed for measuring fractional hemoglobin concentrations of more than two hemoglobin species, the path lengths must be known or estimated at least statistically to enable establishment of calibration for the fractional hemoglobin measurement. The calibration is normally established by collecting a large amount of training data and finding a relationship of the blood hemoglobin fractions, which is determined from blood samples drawn from subjects, and the actual measured signals at the wavelength channels of the measurement device. This relationship is then used as the calibration and it forms a computational model that defines how the final results, i.e. hemoglobin fractions, may be derived from the actual measured signals termed in-vivo measurement signals in this context. Thus, calibration involves determination of a computational model that represents the relationship between the desired blood characteristics and in-vivo measurement signals obtained from the subject. Different types of regression models may be used to accomplish the computational model and thus also calibration.

A major drawback related to the calibration is the vulnerability to errors, which results from the fact that the computational model is obtained for the population average, and therefore, does not involve human individual variability arising from the deviation of the tissue properties from the norm. This leads to compromised accuracy of the device. This is the case regardless of whether a so-called direct or indirect method is used. In the direct method, the concentrations may be determined directly based on the measured signals using population mean calibration coefficients stored during the calibration process, while in the indirect method the concentrations are solved based on a set of equations, where each equation defines a ratio Nⱼₖ=dAⱼ/dAₖ of two modulation ratios

$${dA}_{i} = \frac{A {C}_{i}}{D {C}_{i}} ,$$

where i is the wavelength in question, ACᵢ is the AC component of the plethysmographic signal at wavelength i and DCᵢ is the DC component of the plethysmographic signal at wavelength i. Generally, traditional pulse oximeters try to eliminate the effect of all extrinsic factors, such as the thickness of the finger, on the measurement. Therefore, each signal received is normalized by extracting the AC component oscillating at the cardiac rhythm of the patient, and then dividing the AC component by the DC component of the light transmission or reflection, as indicated above. As mentioned above, most current calibration methods operate under the assumption that the tissue properties remain relatively constant between subjects and within subjects, which leads to the compromised accuracy of the device.

In order to improve the accuracy of pulse oximeters, a method exists which can compensate for human variability in the calibration process. In these pulse oximeters, reference data is stored which is indicative of the calibrating conditions in which the initial calibration takes place. Subject-specific effect on the measurement is taken into account by adding compensation processes that compensate for the human variability and thus create a subject-specific adjustment for the population mean calibration. Separate compensation processes are needed for compensating tissue-induced changes in the efficient wavelengths of the light sources on one hand and in the path lengths of the light beams on the other hand.

Since the compensation for human variability is rather complicated, the overall calibration process also becomes rather complicated. It would therefore be desirable to bring about a calibration mechanism which is as simple as possible but still capable of improving the accuracy of the measurement by reducing the vulnerability to errors induced by human variability.

### BRIEF DESCRIPTION OF THE INVENTION

The above-mentioned problems are addressed herein which will be comprehended from the following specification.

To achieve a solution that combines simple calibration and improved measurement accuracy, calibration is carried out by a computational model that employs, in addition to conventional variables (like ratios Nⱼₖ=dAⱼ/dAₖ), such explanatory variables that are able to provide information on how the real absorption and scattering characteristics of the tissue deviate from the default characteristics that result in the population average calibration coefficients. The said variables are here termed tissue property variables. As discussed below, the model may be introduced in different ways to different types of pulse oximeters.

In an embodiment, a method for calibrating an apparatus intended for non-invasively measuring blood characteristics of a subject comprises providing the apparatus with a computational model representing a relationship between in-vivo measurement signals obtained from the subject and the blood characteristics, wherein the providing includes employing at least one tissue property variable in the computational model, in which the at least one tissue property variable is indicative of absorption and scattering characteristics of the subject's tissue.

In another embodiment, an arrangement for determining blood characteristics of a subject comprises a control and processing unit configured to acquire in-vivo measurement signals from a subject, wherein the control and processing unit is provided with a computational model representing a relationship between the in-vivo measurement signals and desired blood characteristics of the subject and wherein the computational model is adapted to employ at least one tissue property variable indicative of absorption and scattering characteristics of the subject's tissue.

In yet another embodiment, a sensor for an arrangement intended for determining blood characteristics of a subject comprises an emitter unit configured to emit radiation through tissue of the subject at a plurality of measurement wavelengths and a detector unit comprising at least one photo detector adapted to receive the radiation at the plurality of wavelengths and to produce in-vivo measurement signals corresponding to the plurality of measurement wavelengths, wherein the sensor comprises a memory storing an identifier identifying a computational model to be used for determining the blood characteristics, and wherein the computational model is adapted to employ at least one tissue property variable indicative of absorption and scattering characteristics of the subject's tissue.

Various other features, objects, and advantages of the invention will be made apparent to those skilled in the art from the following detailed description and accompanying drawings.

### BRIEF DESCRIPTION OF THE DRAWINGS

FIG. 1 is a block diagram illustrating one embodiment of a multi-wavelength pulse oximeter;

FIG. 2 is a flow diagram illustrating an example of the steps carried in a transformation-based pulse oximeter to obtain blood characteristics of a subject;

FIG. 3 illustrates an example of the operational entities of a processing unit of a pulse oximeter;

FIG. 4 illustrates an example of a pulse oximeter system; and

FIG. 5 is a flow diagram illustrating an example of the steps carried in a pulse oximeter provided with a computational model that does not include transformations.

### DETAILED DESCRIPTION OF THE INVENTION

A pulse oximeter comprises a computerized measuring unit and a sensor or probe attached to the subject, typically to finger or ear lobe of the subject. The sensor includes at least one light source for sending optical signals through the tissue and at least one photo detector for receiving the signal transmitted through or reflected from the tissue. On the basis of the transmitted and received signals, light absorption by the tissue may be determined. During each cardiac cycle, light absorption by the tissue varies cyclically. During the diastolic phase, absorption is caused by venous blood, non-pulsating arterial blood, cells and fluids in tissue, bone, and pigments, whereas during the systolic phase there is an increase in absorption, which is caused by the inflow of arterial blood into the tissue part on which the sensor of the pulse oximeter is attached. Pulse oximeters focus the measurement on this pulsating arterial blood portion by determining the difference between the peak absorption during the systolic phase and the background absorption during the diastolic phase. Pulse oximetry is thus based on the assumption that the pulsatile component of the absorption is due to arterial blood only.

In order to distinguish between two species of hemoglobin, oxyhemoglobin (HbO₂), and deoxyhemoglobin (RHb), absorption must be measured at two different wavelengths, i.e. the sensor of a traditional pulse oximeter includes two different light sources, such as LEDs or lasers. The wavelength values widely used are 660 nm (red) and 940 nm (infrared), since the said two species of hemoglobin have substantially different absorption at these wavelengths. Each light source is illuminated in turn at a frequency which is typically several hundred Hz.

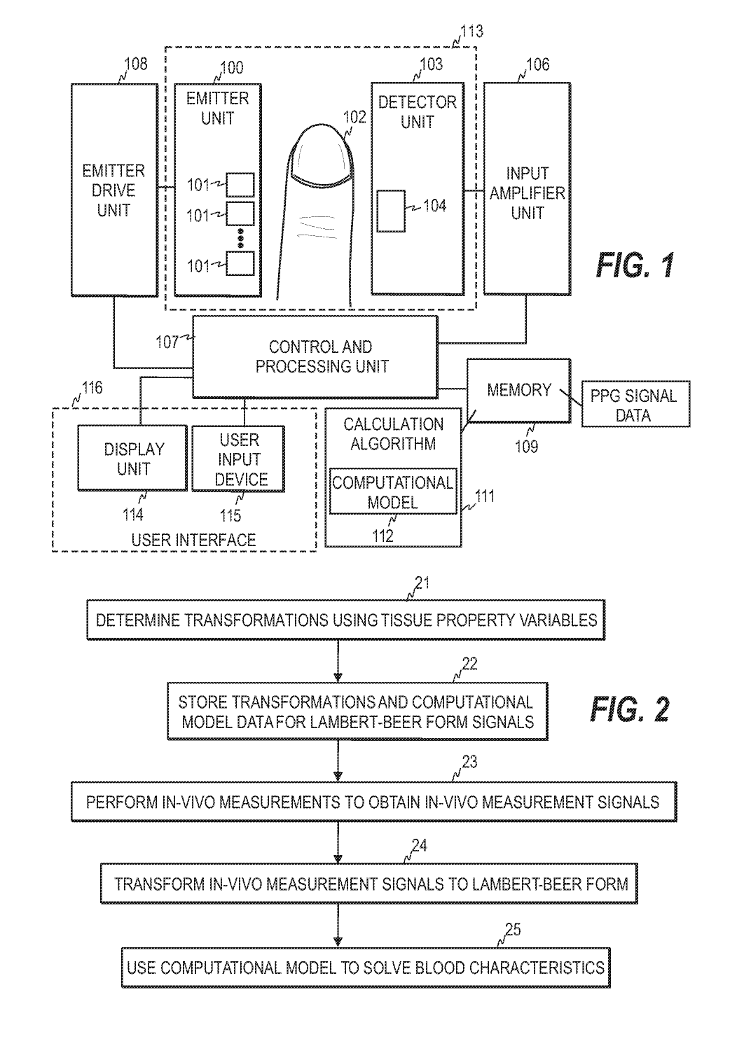

FIG. 1 is a block diagram of one embodiment of a multi-wavelength pulse oximeter. Light transmitted from an emitter unit 100 passes into patient tissue, such as a finger 102. The emitter unit includes multiple light sources 101, such as LEDs, each light source having a dedicated wavelength. Each wavelength forms one measurement channel on which photoplethysmographic waveform data is acquired. The number of sources/wavelengths is at least two and typically between 4 and 8. Below, an example is given in which four wavelengths are used.

The light propagated through or reflected from the tissue is received by a detector unit 103, which comprises one photo detector 104 in this example. The emitter and detector units form the sensor 113 of the pulse oximeter.

The photo detector converts the received optical signals into electrical pulse trains and feeds them to an input amplifier unit 106. The amplified measurement channel signals are further supplied to a control and processing unit 107, which converts the signals into digitized format for each wavelength channel.

The control and processing unit further controls an emitter drive unit 108 to alternately activate the light sources. As mentioned above, each light source is typically illuminated several hundred times per second. With each light source being illuminated at such a high rate compared to the pulse rate of the patient, the control and processing unit obtains a high number of samples at each wavelength for each cardiac cycle of the patient. The value of these samples varies according to the cardiac cycle of the patient, the variation being caused by the arterial blood.

The digitized photoplethysmographic (PPG) signal data at each wavelength may be stored in a memory 109 of the control and processing unit before being processed further by the calculation algorithms of the control and processing unit.

For the determination of blood characteristics, such as oxygen saturation and hemoglobin concentrations, the control and processing unit is adapted to execute a calculation algorithm 111, which may be stored in the memory of the control and processing unit. The obtained blood characteristics and waveforms are shown on the screen of a display unit 114 of a user interface 116, which also includes a user input device 115. The calculation algorithm forms a computational model 112 that represents a (mathematical) relationship between in-vivo measurement signals obtained from the subject and the desired blood characteristics, such as fractional concentrations of different hemoglobin species. The computational model data may be stored in the memory before the pulse oximeter is taken into use. The operations carried out prior to the actual use of the pulse oximeter are in this context referred to as off-line operations, while the actual in-vivo measurements are referred as on-line operations.

As is known, the so-called Lambert-Beer law expresses how light is absorbed by matter. In one embodiment, the pulse oximeter is based on transformations adapted to transform the in-vivo measurement signals obtained from a subject to corresponding non-scatter signals according to the Lambert-Beer model. In a typical transformation-based pulse oximeter, the measured in-vivo signals are first transformed into signals applicable to the Lambert-Beer model and then a linear set of equations applicable in the Lambert-Beer model is solved to obtain the desired blood characteristics, such as the fractional concentrations of different hemoglobin species. An advantage of a transformation-based pulse oximeter is that the transformation does not assume a priori knowledge of the fractional hemoglobin concentrations, since the transformations depend only on total absorption and scattering effect. Therefore, a computational model comprising separate transformations and a set of linear equations is more linear and thus also more accurate than a computational model that determines the blood characteristics directly, i.e. without transforming the in-vivo measurement signals into Lambert-Beer form signals.

Mathematically the operation of a transformation-based pulse oximeter may be expressed as follows: dAᵢ^{LB}=g(dAₖ^{in-vivo},Pₖ), in which dAᵢ^{LB} is a fictitious Lambert-Beer model signal at wavelength i, dAₖ^{in-vivo} is the measured in-vivo signal at wavelength k (k=1 . . . M, in which M is the number of wavelengths), g is a transformation function describing statistically the photon path lengths in the tissue, and Pₖ refers to one or more tissue property variables indicative of absorption and scattering characteristics of the subject's tissue. In the prior art transformation oximetry, the computational model has been obtained without using the tissue property variables Pₖ in the transformation function and searching only the transformation for k=i in the regression analysis.

FIG. 2 illustrates the steps carried out in one embodiment of the pulse oximeter to obtain blood characteristics of a subject. Here, the pulse oximeter is a transformation-based oximeter. First, the off-line operations are carried out in steps 21 and 22. The off-line operations include determination of transformations at step 21 and storing, at step 22, the transformations and the computational model data needed to derive the desired blood characteristics from in-vivo measurement signals converted into Lambert-Beer form signals. Together with the transformations this data forms the computational model that describes the relationship between measured in-vivo signals and the desired blood characteristics. When the pulse oximeter is taken into use, on-line measurements are performed to obtain in-vivo measurement signals dAₖ^{in-vivo} from a subject (step 23). As discussed below, this step involves determination of the variables, such as the tissue property variables, of the model. The in-vivo measurement signals obtained are then first transformed to Lambert-Beer form signals using the transformations stored in the pulse oximeter (step 24). The transformations depend on the tissue property variables, for example. The transformed signals dAᵢ^{LB} are then used according to the stored computational model data to obtain the blood characteristics of the subject (step 25). In case of different haemoglobin species, this involves solving a linear set of equations (dAᵢ^{LB})=c*(εᵢⱼ)(HbXⱼ), in which HbXⱼ is a hemoglobin fraction, the summation is over index j and εᵢⱼ is the extinction coefficient for the analyte j at wavelength i. In a four-wavelength system, for example:

$$( \begin{matrix}{dA}_{1}^{LB} \\ {dA}_{2}^{LB} \\ {dA}_{3}^{LB} \\ {dA}_{4}^{LB}\end{matrix} ) = {C}^{*} ( \begin{matrix}{ɛ}_{1}^{HbO 2} & {ɛ}_{1}^{Hb} & {ɛ}_{1}^{HbCO} & {ɛ}_{1}^{HbMet} \\ {ɛ}_{2}^{HbO 2} & {ɛ}_{2}^{Hb} & {ɛ}_{2}^{HbCO} & {ɛ}_{2}^{HbMet} \\ {ɛ}_{3}^{HbO 2} & {ɛ}_{3}^{Hb} & {ɛ}_{3}^{HbCO} & {ɛ}_{3}^{HbMet} \\ {ɛ}_{4}^{HbO 2} & {ɛ}_{4}^{Hb} & {ɛ}_{4}^{HbCO} & {ɛ}_{4}^{HbMet} \end{matrix} ) * ( \begin{matrix}Hb O 2 \\ Hb \\ Hb CO \\ HbMet\end{matrix} ) , \qquad ( Eq . 1 )$$

where C is a constant, εᵢ^{HbO2} is the extinction coefficient of oxyhemoglobin at wavelength i (i=1, . . . , 4), εᵢ^{RHb} is the extinction coefficient of deoxyhemoglobin at wavelength i, εᵢ^{HbCO} is the extinction coefficient of carboxyhemoglobin at wavelength i, εᵢ^{HbMet} is the extinction coefficient of methemoglobin at wavelength i, and HbO2, RHb, HbCO, and HbMet are the concentrations of oxyhemoglobin, deoxyhemoglobin, carboxyhemoglobin, and methemoglobin, respectively. By measuring four in-vivo signals dAₖ^{in-vivo} (k=1, . . . , 4) and converting them into Lambert-Beer form signals dAᵢ^{LB} (i=1, . . . , 4), the concentrations may be determined based on Equation 1.

When there are four unknown haemoglobin concentrations and four measured signals, Eq. 1 can be solved by inverting the extinction matrix, which gives the concentrations within the constant C, i.e. the relative concentration fractions: C*(HbXⱼ)=(εᵢⱼ)⁻¹*(dAᵢ^{LB}). When there are more than four wavelengths and thereby more than four measured signals and still only four unknown haemoglobin concentrations, the concentration fractions must be solved, for instance, in the least square sense: C*(HbXⱼ)=(εᵢⱼ^{T}εᵢⱼ)⁻¹(εᵢⱼ^{T})*(dAᵢ^{LB}), in which the εᵢⱼ^{T} is the transpose of the extinction matrix εᵢⱼ, i=1 . . . M, and j=1 . . . 4, in which M>4 is the number of wavelengths.

As discussed above, the transformations are determined in step 21. This typically involves determination of a plurality of transformation coefficient sets, each set comprising coefficients for the independent variables, i.e. explanatory variables, of the regression model. In a pulse oximeter provided with M wavelengths, M or M−1 sets of coefficients need to be determined depending on whether the blood characteristics are calculated based on modulation signals dAᵢ^{LB} or ratios of the modulations signals Nᵢⱼ^{LB}=dAᵢ^{LB}/dAⱼ^{LB}. In one embodiment of the pulse oximeter, the determination of the transformations may be carried out in the following way. The pulse oximeter signals are measured from a subject at a stable blood concentration level. An arterial blood sample is then drawn from a subject and the hemoglobin concentrations (HbO2, RHb, HbCO, and HbMet) are determined from the blood sample using a reference CO-oximeter. These concentrations are then set into Eq. 1. The extinction coefficients in the extinction matrix are obtained from tabulated blood data literature at the light source wavelengths used in the oximetry probe. The Lambert-Beer signals dAᵢ^{LB} can now be calculated using Eq. 1. The measured dAₖ^{in-vivo}, k=1 . . . M, the measured tissue property variables Pₖ, k=1 . . . N, at the blood sample draw and the corresponding calculated dAᵢ^{LB} are stored for later analysis. The above-described process is then repeated at different levels of blood concentrations for many subjects until a pre-set statistical power is reached. In a conventional prior art transformation oximetry, the transformation function is determined by regression of the measured Nᵢⱼ^{in-vivo}=dAᵢ^{in-vivo}/dAⱼ^{in-vivo} with the corresponding Nᵢⱼ^{LB}=dAᵢ^{LB}/dAⱼ^{LB} i.e. only the two Nᵢⱼ's are regressed. However, in the pulse oximeter of FIG. 2, a regression model is searched between each Nᵢⱼ^{LB} and all the measured Nᵢⱼ^{in-vivo} (i,j=1 . . . M) and all the tissue property variables Pₖ, k=1 . . . N.

The tissue property variables may include variables such as relative light transmission, i.e. ratio DCₖₗ of the DC signals normalized to describe the relative light transmission through tissue at two wavelengths k and l, subject pulsation strength, i.e. perfusion index, depth of the respiratory modulation in the plethysmographic signals, etc. Typically, the regression model also includes square terms or higher of each possible independent variable and their cross terms, such as Nᵢⱼ×Nₖₗ or Nᵢⱼ×DCₖₗ. The coefficients of the regression model are stored in the memory 109 or in the calculation algorithm 111. The regression model typically includes 4 to 20 different variables and their derivatives.

The optimal regression model for each dependent variable, for instance for each Nᵢⱼ^{LB}, can be found by first tabulating at each calibration point, i.e. at each arterial blood draw during step 21, the dependent variable and all the independent variables into a table. The table for N₁₄^{LB}, for example, may look as follows:

TABLE 1

| | | | | | | |
|---|---|---|---|---|---|---|
| | N₁₄^{LB}, | | | | All cross | |
| Blood | dependent | | | | terms for | All Pₖ and other |
| Draw# | variable | N₁₂^{in-vivo} | N₁₃^{in-vivo} | N₁₄^{in-vivo} | Nᵢⱼ^{in-vivo} | in-vivo terms |

1
2
. . .
Last

The regression model for the dependent variable N₁₄^{LB} now be searched for by using known mathematical methods. For instance, in a so-called forward selection method, the most significant explaining parameter (one of the columns in the table) is found first by picking variables one by one from the table and minimizing the error between the dependent and the one independent variable. Then the next significant explaining term can be found by minimizing the remaining residual error between the dependent variable and the two best picked independent variables from the table. This process is continued until all significant (all explaining and none redundant) terms are included in the model, the residual error reaches a pre-set limit or the residual error cannot be further reduced. Other mathematical methods, such as backward elimination method, stepwise method, principal component analysis or factor analysis, may also be used for searching for the best regression model.

As discussed above, step 22 involves the determination of the parameters and equations that belong to the computational model besides the transformations, i.e. the data needed to derive the desired blood characteristics from signals converted to be compatible with the Lambert-Beer model. The computational model also forms the calibration for the desired blood characteristics, such as fractional hemoglobin concentrations. In the example of Equation 1, the data stored in the pulse oximeter in step 22 may thus include 3 or four sets of transformation coefficients, each set comprising a coefficient for each independent variable of the regression model, 16 extinction coefficients, and the actual equations through which the concentrations may be determined, i.e. equations that indicate the relationship between the desired blood characteristics and determined Nᵢⱼ^{LB} or dAᵢ^{LB} values.

In step 23, the in-vivo signals are measured. In addition to the actual modulation signals dAᵢ^{in-vivo} (i=1, . . . , M), all independent variables of the regression model, such as the tissue property variables Pₖ, are derived from the plethysmographic waveforms. For each cardiac pulse and each wavelength the DCi values that correspond to the maximum light transmission during the cardiac pulse are determined. Similarly, all other tissue property variables are determined cardiac cycle by cycle. For instance, the strength of the pulsation may be determined on one of the wavelength channels at or near the isobestic absorption wavelength of the oxy- and deoxyhemoglobins. An indicator for the pulsation strength may thus be the amplitude, i.e. the cardiac modulation depth, of the plethysmographic waveform at or near 800 nm, that is the difference of the peak and valley values of the PPG waveform during one cardiac cycle.

In step 24, respective transformation coefficients stored in step 21 are retrieved for the determined in-vivo variables and the Lambert-Beer modulations signals dAᵢ^{LB} or alternatively the ratio of the Lambert-Beer modulation signals Nij^{LB}=dAi^{LB}/dAj^{LB}, depending how the regression model has been constructed, are calculated. In step 25, the blood characteristics are calculated by solving the stored equations between the Lambert-Beer signals and the desired blood characteristics, such as Eq. 1, to obtain the analyte concentrations.

As the tissue property variables are employed in the regression model, the calibration mechanism can take human variability into account and is therefore less prone to errors than the conventional mechanisms that are based on a default, non-variant tissue structure.

Table 2 below shows an example of one set of transformation coefficients determined in step 21 in a pulse oximeter comprising 8 wavelengths (such as 612 nm, 632 nm, 660 nm, 690 nm, 725 nm, 760 nm, 800 nm, and 900 nm). This example shows the set of coefficients for a transformation g17, i.e. the set is used to obtain N₁₇^{LB}.

TABLE 2

| Term | Transformation coefficient |
|---|---|
| Constant | 3.94 |
| N₁₇ | 0.19 |
| N₃₇ | 1.02 |
| N₅₇ | −1.41 |
| DC₁₇ | 44.45 |
| DC₂₇ | −21.77 |
| DC₃₇ | 5.83 |
| DC₅₇ | −3.49 |
| PI(7) | 0.50 |
| N₁₇ × N₁₇ | −0.88 |
| N₃₇ × N₃₇ | 0.91 |
| DC₂₇ × DC₃₇ | −15.17 |
| DC₂₇ × DC₅₇ | 17.06 |

Thus, in this example transformation g17 is used to obtain N₁₇^{LB}=dA₁^{LB}/dA₇^{LB} as follows: N₁₇^{LB}=3.94+0.19×N₁₇^{in-vivo}+1.02×N₃₇^{in-vivo}−1.41×N₅₇^{in-vivo}+44.45×DC₁₇−21.77×DC₂₇+5.83×DC₃₇−3.49×DC₅₇+0.50×PI(7)−0.88×N₁₇×N₁₇+0.91×N₃₇×N₃₇−15.17×DC₂₇×DC₃₇+17.06×DC₂₇×DC₅₇.

Here, variables DC₁₇, DC₂₇, DC₃₇, and DC₅₇ are the above-described tissue property variables indicative of relative light transmission. PI is the perfusion index, i.e. the plethysmographic modulation depth in percents, and PI(7) is the perfusion index at the seventh wavelength (800 nm).

As mentioned above, the number of transformation coefficient sets to be determined depends on the number of wavelengths; M−1 sets are needed if Nᵢⱼ^{LB} values are calculated and M sets are needed if dAᵢ^{LB} values are calculated, where M is the number of wavelengths. Consequently, in the above example, 7 coefficient sets may be determined. Thus, in addition to g17, 6 other coefficient sets are determined, that is g27, g37, g47, g57, g67, and g87 (g77=1).

Above, transformation oximetry has been presented in the form that the in-vivo measured signals are transformed to the theoretical signals in the non-scatter Lambert-Beer tissue model. However, the transformation may also be applied to the extinction coefficients that change, under tissue interaction, from the ideal non-scatter Lambert-Beer extinction coefficients to effective in-vivo extinction coefficients that correspond to a characteristic photon path length on each wavelength channel. In this presentation Eq. 1 may be written to read:

$$( \begin{matrix}{dA}_{1}^{i n - vivo} \\ {dA}_{2}^{i n - vivo} \\ {dA}_{3}^{i n - vivo} \\ {dA}_{4}^{i n - vivo}\end{matrix} ) = {C}^{*} ( \begin{matrix}L 1 * {ɛ}_{1}^{HbO 2} & L 1 * {ɛ}_{1}^{Hb} & L 1 * {ɛ}_{1}^{HbCO} & L 1 * {ɛ}_{1}^{HbMet} \\ L 2 * {ɛ}_{2}^{HbO 2} & L 2 * {ɛ}_{2}^{Hb} & L 2 * {ɛ}_{2}^{HbCO} & L 2 * {ɛ}_{2}^{HbMet} \\ L 3 * {ɛ}_{3}^{Hb O 2} & L 3 * {ɛ}_{3}^{Hb} & L 3 * {ɛ}_{3}^{HbCO} & L 3 * {ɛ}_{3}^{HbMet} \\ L 4 * {ɛ}_{4}^{HbO 2} & L 4 * {ɛ}_{4}^{Hb} & L 4 * {ɛ}_{4}^{HbCO} & L 4 * {ɛ}_{4}^{HbMet}\end{matrix} ) * ( \begin{matrix}Hb O 2 \\ Hb \\ Hb CO \\ HbMet\end{matrix} ) \qquad ( Eq . 2 )$$

in which signals dAᵢ^{in-vivo} are the in-vivo measured modulation ratios and L1 . . . L4 are path length multipliers, corresponding to effective photon path lengths in the in-vivo tissue. The path length multipliers thus constitute an extinction matrix transformation that defines the effective extinction matrix in in-vivo tissues. The hemoglobin fractions HbO2, Hb, HbCO and HbMet can now be solved by inverting the effective in-vivo extinction matrix and multiplying the measured modulation ratio signals by this matrix.

In one embodiment of the pulse oximeter arrangement, the regression models are searched for for the path length multipliers L1 to L4 or for their ratios L1/L4, L2/L4, and L3/L4. This approach has the advantage that it incorporates the wavelength shift of the LED emission in the regression model without an additional step needed to compensate for the shift, as the relative tissue transmissions DCᵢ/DCⱼ will at any rate affect the effective wavelengths of each measurement channel, and thereby, the Lambert-Beer extinction coefficients defined at the emission center wavelength of the LED in Eq. 1. The regression model for the effective path lengths Lᵢ may be searched for according to the following equation:

Lᵢ=G(dAₖ^{in-vivo},Pₖ)

in which the function G is dependent on the actual measured signals dAₖ^{in-vivo} and the tissue property variables Pₖ, similarly as the transformation function g above. Consequently, in a transformation-based arrangement, the transformation rules that take into account the effect of in-vivo tissue on the measurement signals may be determined in various ways.

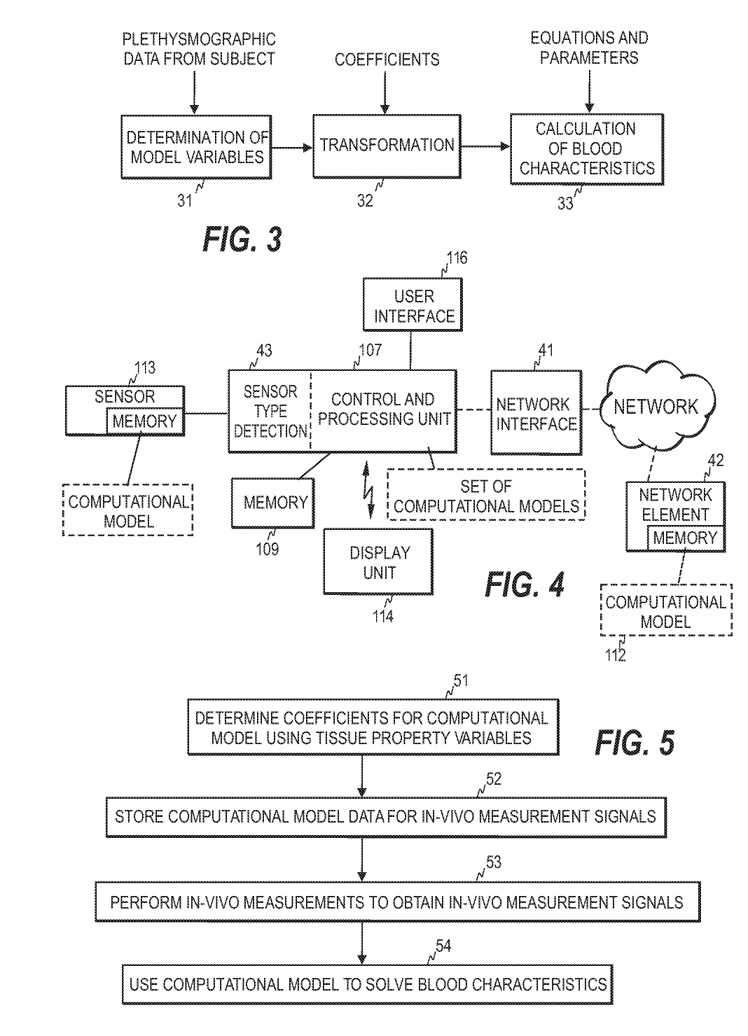

In terms of the calculation process, the functionalities of the control and processing unit 107 may be divided into the operational units shown in FIG. 3. A variable determination unit 31 is configured to determine the values of the regression model variables based on the measured in-vivo signals and a transformation unit 32 is configured to carry out the transformations, thereby to transform the in-vivo measurement signals to signals compatible with the Lambert-Beer model or to obtain path length multipliers (or path length multiplier ratios). A calculation unit 33 is further configured to calculate the desired blood characteristics based on the transformed variables output from the transformation unit. It is to be noted that FIG. 3 illustrates the division of the functionalities of the control and processing unit in logical sense and in terms of blood parameter calculation. In a real apparatus the functionalities may be distributed in different ways between the elements or units of the apparatus or system.

The above mechanism allows a rather simple calibration process to be combined with increased accuracy, since the modeled transformations form an engine that describes the blood characteristics without a need to carry out plenty of additional measurements on the subject to improve the accuracy of the measurement and without a need to store the initial calibration conditions in the pulse oximeter. Many of the tissue property variables may be determined directly from the plethysmographic waveforms without a need for separate measurements. The approach conforms to the traditional calibration in the sense that the computational model describes the relationship between the desired blood characteristics and the in-vivo measurement signals. However, the approach deviates from traditional calibration in the sense that the model does not assume substantially constant tissue properties between subjects and within subjects.

The pulse oximeter of FIG. 1 includes hemoglobin and/or SpO2 calculation algorithm(s) that may be stored in the memory of the pulse oximeter in the manufacturing phase of the apparatus. As discussed above, the calculation algorithm(s) form the computational model that employs tissue property variables and thus also includes coefficients for the tissue property variables determined in on-line measurements. However, it is to be noted that the elements of computational model, i.e. variables and equations, are not necessarily stored in the actual pulse oximeter or in its control and processing unit, but the elements of the computational model may be distributed between the sensor attached to the subject, the actual pulse oximeter device, i.e. the control and processing unit, and/or a communication network. Thus, a complete pulse oximeter may be realized as a compact or distributed device. Below, the term arrangement is used to refer to the multiple possible device implementations in this respect. FIG. 4 illustrates an example of an arrangement in which the control and processing unit 107 is provided with a network interface 41 for downloading/updating the computational model or components thereof through a network from a network element 42 storing the model or components thereof, such as updated coefficients. This is illustrated with dotted lines in the figure. The computational model 112 or the components thereof may also be stored in the sensor 113, as is illustrated in FIG. 4. The control and processing unit may also hold several computational models for different sensor types. The sensors may be provided with an identifier identifying the computational model(s) that may be used with the sensor. In one embodiment of the arrangement, the control and processing unit is compatible with both a conventional sensor (two wavelengths) and an advanced sensor employing tissue-related variables in the computational model. The control and processing unit 107 may be provided with a recognition module 43 for recognizing the type of the sensor and reading the model identifier. If the recognition module detects that an advanced sensor is connected to it, it may download data from the sensor and/or network according to the blood characteristics to be determined and displayed. The user of the device may select the data to be displayed through the user interface 116.

In the examples of Eq. 1, the computational model includes the transformations that convert the in-vivo measurement signals into fictitious Lambert-Beer signals. Although the transformation-based computational model has the above-mentioned advantages, the computational model may also solve the concentrations directly, i.e. the computational model may be a regression model of the form HbXk=h(Nij, Pk), where HbXk is the concentration to be solved. FIG. 5 is a flow diagram illustrating the steps carried out in this embodiment. In step 51, regression model coefficients are determined using tissue property variables in the model. The dependent variable now is a concentration fraction of a hemoglobin derivative, which can be HbO2, RHb, HbCO or HbMet. Thus, in case of these four hemoglobin derivatives, four different models are always constructed, regardless of the number of wavelengths used in the pulse oximeter. The modeling process is the same as for the transformations, and again different known mathematical optimization methods may be used to search for the optimal regression model. In step 52, the regression model data is stored in the pulse oximeter. Steps 51 and 52 are off-line steps in which the pulse oximeter is provided with a computational model that represents the relationship between in-vivo measurement signals and the desired blood characteristics. When the pulse oximeter is taken into use, on-line measurements are performed to obtain in-vivo measurement signals from a subject (step 53). The measured in-vivo variables are then used according to the stored computational model data to obtain the blood characteristics of the subject (step 54). The embodiment of Eq. 2 may be regarded as a combination of the direct and transformation-based solutions, since in this embodiment the transformations are applied to the extinction coefficients through the effective path lengths Lᵢ, and yet the concentrations may be solved directly according to Eq. 2.

In the above examples, the pulse oximeter is provided with at least four wavelengths for determining fractional hemoglobin concentrations. However, the pulse oximeter may also be a conventional two-wavelength pulse oximeter in which the SpO₂ calculation algorithm utilizes the tissue property variables. In a conventional pulse oximeter, the SpO₂ value is typically calculated according to the equation: SpO₂=C0−C1×N₁₂−C2×N₁₂×N₁₂, where N₁₂=dA₁/dA₂ and C0-C2 are the coefficients of the computational model. If tissue property variables are employed in this kind of a pulse oximeter, the said novel variables may be added to the above computational model. For example, three new terms may be added to the computational model as follows: SpO₂=C0−C1×N₁₂−C2×N₁₂×N₁₂+A1×(DCₗ/DC₂)+A2×(DC₁/DC₂)×(DC₁/DC₂)+A3×N₁₂×(DC₁/DC₂), where A1-A3 are the coefficients of the tissue property variables. Thus, the new terms may be regarded as corrections to the nominal calibration. Applying this to FIG. 5, coefficients C0-C2 and A1-A3 are determined in step 51, while the actual model data. i.e. the coefficients and the equation yielding the SpO2 value, is stored in step 52.

Consequently, the pulse oximeter is provided with regression models in which the tissue property variables serve as independent variables. In a transformation-based pulse oximeter the regression models may be applied to the transformations, while in a pulse oximeter configured to determine the blood characteristics directly the computational model may itself correspond to the optimal regression model comprising tissue property variables as independent variables. As discussed above, combinatory embodiments that combine transformations with a direct solution model are also possible.

A pulse oximeter may also be upgraded to a device capable of determining the concentration of a substance in the blood of a patient. Such an upgrade may be implemented by delivering to the pulse oximeter a software module that enables the device to carry out the above steps. The software module may be delivered, for example, on a data carrier, such as a CD or a memory card, or through a telecommunications network. The software module may be provided with the computational model or model elements and/or with operational entities adapted to access an external memory holding the model or model elements, such as model coefficients.

This written description uses examples to disclose the invention, including the best mode, and also to enable any person skilled in the art to make and use the invention. The patentable scope of the invention is defined by the claims, and may include other examples that occur to those skilled in the art. Such other examples are intended to be within the scope of the claims if they have structural or operational elements that do not differ from the literal language of the claims, or if they have structural or operational elements with insubstantial differences from the literal language of the claims.

1. A method for calibrating an apparatus intended for non-invasively measuring blood characteristics of a subject, the method comprising providing the apparatus with a computational model representing a relationship between in-vivo measurement signals obtained from the subject and the blood characteristics, wherein the providing includes employing at least one tissue property variable in the computational model, in which the at least one tissue property variable is indicative of absorption and scattering characteristics of the subject's tissue.

2. The method according to claim 1, wherein the providing includes determining transformation rules indicative of how actual photon path lengths in the subject's tissue affect the in-vivo measurement signals.

3. The method according to claim 2, wherein the determining the transformation rules includes defining transformation coefficient sets, each transformation coefficient set being defined based on a regression model, where the at least one tissue property variable serves as an independent variable.

4. The method according to claim 2, wherein the providing further includes storing computational model data that indicate a relationship between theoretical Lambert-Beer measurement signals and the blood characteristics.

5. The method according to claim 1, wherein the providing includes employing the at least one tissue property variable in the computational model, in which the at least one tissue property variable includes at least one variable from a set of variables including a variable indicative of relative light transmission, a variable indicative of perfusion index and a variable indicative of depth of respiratory modulation.

6. The method according to claim 1, wherein the providing comprises providing the apparatus with the computational model, in which the computational model forms a regression model, where the at least one tissue property variable serves as an independent variable.

7. An arrangement for determining blood characteristics of a subject, the arrangement comprising a control and processing unit configured to acquire in-vivo measurement signals from a subject, wherein the control and processing unit is provided with a computational model representing a relationship between the in-vivo measurement signals and desired blood characteristics of the subject and wherein the computational model is adapted to employ at least one tissue property variable indicative of absorption and scattering characteristics of the subject's tissue.

8. The apparatus according to claim 7, wherein the computational model comprises predetermined transformation rules indicative of how actual photon path lengths in the subject's tissue affect the in-vivo measurement signals.

9. The arrangement according to claim 8, wherein the transformation rules include transformation coefficient sets, each transformation coefficient set belonging to a regression model, where the at least one tissue property variable serves as an independent variable.

10. The arrangement according to claim 8, wherein the computational model further comprises computational model data that indicate a relationship between theoretical Lambert-Beer measurement signals and the blood characteristics.

11. The arrangement according to claim 7, wherein the at least one tissue property variable includes at least one variable from a set of variables including a variable indicative of relative light transmission, a variable indicative of perfusion index and a variable indicative of depth of respiratory modulation.

12. The arrangement according to claim 11, wherein the variable indicative of relative light transmission represents a ratio of DC signal levels at two wavelengths.

13. The arrangement according to claim 7, wherein the computational model is formed by a regression model, where the at least one tissue property variable serves as an independent variable.

14. A sensor for an arrangement intended for determining blood characteristics of a subject, the sensor being attachable to the subject and comprising:
an emitter unit configured to emit radiation through tissue of the subject at a plurality of measurement wavelengths;
a detector unit comprising at least one photo detector adapted to receive the radiation at the plurality of wavelengths and to produce in-vivo measurement signals corresponding to the plurality of measurement wavelengths,
wherein the sensor comprises a memory storing an identifier identifying a computational model to be used for determining the blood characteristics, wherein the computational model is adapted to employ at least one tissue property variable indicative of absorption and scattering characteristics of the subject's tissue.
